(19) United States
(12) Patent Application Publication
(10) Pub. No.: US 2022/0071497 A1
(43) Pub. Date: Mar. 10, 2022

(54) VASCULAR ASSESSMENT USING ACOUSTIC SENSING

(71) Applicant: General Electric Company, Schenectady, NY (US)

(72) Inventors: Prem Venugopal, Clifton Park, NY (US); Peter Michael Edic, Albany, NY (US); Thomas Kwok-Fah Foo, Clifton Park, NY (US)

(21) Appl. No.: 17/422,653

(22) PCT Filed: Jan. 16, 2020
(86) PCT No.: PCT/US2020/013935

§ 371 Date: Jul. 13, 2021

(51) Int. Cl. A61B 5/02; A61B 5/00; G16H 50/20; G16H 50/70; G16H 50/30; G16H 30/40; G06T 7/00

(52) U.S. Cl. CPC A61B 5/02028; A61B 5/02007; A61B 5/746; A61B 5/0095; G16H 50/20; G16H 50/70; G06T 2207/10081; G16H 30/40; G06T 7/0012; G06T 2207/30101; G06T 2207/10132; G06T 2207/10116; G06T 2207/10088; G16H 50/30

(57) ABSTRACT

The present disclosure relates to the use of prior images acquired of the patient and acoustic signature from a vascular region of interest to create a patient-specific model of sound propagation from the vascular region. This model is then used to monitor the progression of disease in the vascular region of interest, using subsequently-acquired acoustic signals. In an alternate embodiment, population-based images and/or population-based acoustic signatures are used to generate predictive data when a priori patient-specific imaging information is not available and this data is used to characterize or categorize at-risk patients suspected of coronary artery disease, but without prior cardiac events.

### CROSS-REFERENCE TO RELATED APPLICATION

This application claims priority to and the benefit of Provisional Patent Application No. 62/793,278, entitled “VASCULAR ASSESSMENT USING ACOUSTIC SENSING”, filed Jan. 16, 2019, which is herein incorporated by reference in its entirety.

### BACKGROUND

The subject matter disclosed herein relates to the use of acoustic sensing in the context of vascular assessment.

In many instances it may be desirable to acquire vascular information about a patient either based on prior events or treatments or as a way to monitor or predict cardiovascular and heart health, or the hemodynamic significance of a vascular anomaly. In general, however, techniques providing the most detailed or comprehensive information are the most time intensive or involved, potentially involving a lengthy scan or procedure and/or subjecting the patient to an imaging protocol that may also involve the use of sedation, exogeneous contrast agents, or other techniques which add to the complexity and invasiveness of the procedure. Moreover, in some instances, such as early onset of disease, techniques may also increase the overall cost for managing the patient.

Conversely, those techniques that are less involved may, correspondingly, provide less useful or comprehensive information. For example, techniques that do not image the heart or vasculature in some manner may fail to provide useful structural detail or information that is needed to obtain an accurate assessment of a patient's current vascular health or is inadequate for use in predicting future vascular health. Alternatively, less-complex imaging methods that provide quantitative information about the hemodynamic significance of vascular anomalies, such as doppler ultrasound, may be used but all imaging techniques require a greater degree of complexity and training on the part of the operator, or require more sophisticated medical equipment. This makes continuous monitoring not feasible as the imaging methods require separate equipment and may not be necessarily available in any setting, especially outside a medical treatment facility.

### BRIEF DESCRIPTION

Certain embodiments commensurate in scope with the originally-claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

The present disclosure relates to the use of acquired acoustic signals in a vascular monitoring or assessment context. In various described implementations, the acquired acoustic signals (e.g., acoustic spectral signals—frequency-dependent energy content in the acoustic signal—or acoustic temporal signals) for a patient may be used to assess a current vascular state of the patient or assess or predict future vascular characteristics of the patient. In certain implementations, the current acoustic signals are used in conjunction with a model or other data construct that relates image or other structural and/or functional vascular data to corresponding acoustic signals. In this manner, the current acoustic signal may be related to a structural and/or functional assessment of the vasculature, which may be used to assess a current patient state or to assess the risk for future cardiovascular events.

The present disclosure also teaches that for a specific patient, the changes in acoustic spectral signals can be better predicted if coupled with prior imaging data that characterize the vascular region and associated collateral vascular structures relevant to the site of the vascular anomaly, and a baseline acoustic spectral measurement. Subsequent to this, changes in the vascular anomaly, i.e., changes in vessel cross-sectional area or regurgitant volume in the case of valvular anomalies, can be modeled and the resulting changes in the acoustic spectral signal can be predicted and matched with the physical change in the vascular anomaly, and the associated hemodynamic significance. As such, any measured acoustic spectral signal can be matched to the modeled acoustic spectral signature to provide an accurate assessment of the change in hemodynamic significance of the progression or regression of vascular disease without enduring multiple diagnostic imaging sessions. Furthermore, this approach, after acquiring a baseline examination to provide the prior imaging data for information on the vascular structure and hemodynamic vascular data, generates a patient-specific library of possible changes as a function of vascular anomalies. This allows at any point in time, comparison of the present acoustic spectral signature with that of the patient-specific library to determine the severity of the vascular anomaly in a rapid and simple manner without requiring additional complex imaging

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a block diagram of an acoustic sensing system, in accordance with aspects of the present disclosure;

FIG. 2 depicts an example of a process flow for estimating or characterizing a vasculature defect or structural change using observed changes in sound spectra, in accordance with aspects of the present disclosure;

FIG. 3 depicts an example of a process flow for classifying or stratifying patient risk with respect to future cardiovascular events, in accordance with aspects of the present disclosure; and

FIG. 4 depicts an example of a process flow for monitoring a patient for present or prospective cardiovascular events, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

As discussed herein, assessment of current or predicted vascular conditions using acoustic signals is described. In accordance with the techniques discussed herein the acoustic signals may be used in conjunction with models or constructs generated based on prior acoustic, structural, and hemodynamic data (e.g., from X-ray-based angiography data, computed tomography data, magnetic resonance data, ultrasound data, population models, and so forth, and combinations thereof) to predict the acoustic spectral signature corresponding to a current or future vascular prognosis for a patient. With this in mind, certain specific use cases may be described herein to provide real-world contexts and to simplify explanation. It should be understood, however, that the examples discussed herein are provided merely to illustrate and provide context for the present techniques, and should not be construed as limiting. Specifically, although the techniques are described in the context of assessing patency of coronary arteries, the techniques are applicable to vascular disease in other contexts, e.g., peripheral cardiovascular disease, valvular disease, cardiovascular disease of non-cardiac organ systems, and so forth.

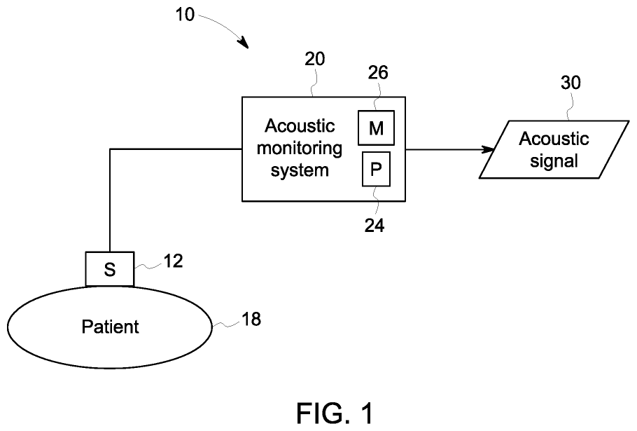

With the preceding in mind, and by way of providing useful context, FIG. 1 depicts a high-level view of components of an acoustic sensing system 10 that may be employed in accordance with the present approach. The illustrated acoustic sensing system 10 includes a sensor 12 or probe suitable for contact with a subject or patient 18 during an examination. By way of example, the sensor 12 may comprise one or more microphones or other type of transducers (including multiple or arrays of transducers) capable of converting skin vibrations to acoustic or digital electrical signals for downstream processing. Note that the microphones described need not necessarily have the capability of imaging, as in ultrasound transducer probes. It is noted that ultrasound transducer probes have the capability of detecting and recording acoustic spectral signals but this invention teaches a method that does not require the capability of imaging to determine the hemodynamic significance of the vascular anomaly. Thus, the process for monitoring for changes is vastly simplified if concurrent imaging is not necessary. Unlike that depicted in FIG. 1, the acoustic sensing system can also be a non-contact device, such as a laser doppler vibrometer. In addition, the present approach envisions that one could design an acoustic sensing system that is capable of detecting vibrations below the skin surface.

In the depicted example, the sensor 12 is in communication with an acoustic monitoring system 20. The depicted example depicts a physical or wired connection, though it should be understood that the sensor 12 and acoustic monitoring system may also communicate wirelessly. A processing component 24 (e.g., a microprocessor) may be used to execute stored routines (such as routines stored in a memory 26, to process data acquired using the sensor 12 and to output an acoustic signal 30 (e.g., an acoustic spectral signature or sound spectrum) representative of the acquired acoustic data. Acoustic signal 30 may be analyzed directly or compared to longitudinal data and/or data generated using one or more models of vasculature and surrounding environment.

With the preceding system level discussion in mind as useful context, the present techniques may utilize such an acoustic sensing system 10 to acquire acoustic or sound data that may be used in the evaluation of vasculature of a patient. As noted above, though the present discussion may utilize a specific example to provide context, the present techniques should be understood to be generally applicable to detecting or predicting a range of (if not all) vascular defects or irregularities based on acoustic signature data.

One implementation of the current technique, unlike prior techniques, uses images previously acquired of the patient and an acoustic signature (e.g., an acoustic signature acquired contemporaneously or close to contemporaneously with imaging data in the example below) to create a patient-specific model of sound propagation from vasculature of interest (e.g., the coronary arteries). This model may then be subsequently used to monitor the progression of a vascular disease, such as to determine the degree of stenosis or re-stenosis, using subsequently acquired acoustic signals. In a further aspect, population-based images may be used to generate predictive data when prior, patient-specific imaging information is not available. Such an approach may be useful for risk stratification of asymptomatic patients at risk for coronary artery disease, but without prior history of cardiovascular disease or cardiac events for which image data may be obtained.

To provide one example, the present technique may be useful in evaluating a patient for occurrence of re-stenosis. By way of background, stents are small wire meshes used to invasively treat arterial blockages. To deploy a stent, a catheter is inserted into a blocked artery during an interventional procedure, typically under X-ray guidance. Angioplasty is used to reestablish blood flow and the stent is then deployed within the location of the previous blockage, thereby allowing blood to pass more freely.

However, over time, the artery may become blocked again due to excessive tissue growth and/or plaque deposition around the stent. This is referred to as a re-stenosis. In the case of the coronary arteries, re-stenosis can lead to recurrence of previously alleviated symptoms, such as angina, indicating that the blood flow to the heart is not sufficient to supply the patient's needs. The patient then may need to undergo another interventional procedure to assess vessel patency and/or again open the blocked artery.

Current clinical guidelines recommend invasive coronary angiography for symptomatic patients to check for the presence of re-stenosis. Approximately, 900,000 coronary interventions involving stent placement are performed yearly in the US and re-stenosis rates of 12% to 30% have been reported. The average cost of a coronary angiogram to check for the presence of re-stenosis is around $3000, not to mention the risks associated with this invasive procedure, and the additional contrast and radiation dose to the patient. Thus, a low-cost method to non-invasively evaluate a stented patient, without subjecting the patient to additional radiation and contrast burden, may be of economic and clinical value.

With this in mind, one implementation of the present technique uses previously-acquired X-ray images acquired in the guided procedure for stent deployment, in conjunction with acoustic data, to assess the health of the patient's vasculature, such as the likelihood or extent of re-stenosis. In some cases, X-ray images (or additional X-ray images) may be obtained after the stent placement and such images can also be utilized. In addition to X-ray images obtained during an interventional procedure, suitable images for the present technique may also be obtained using Computed Tomography Angiography (CTA) (such as prior to stent placement), Magnetic Resonance Angiography (MRA), phase-contrast Magnetic Resonance Imaging (MRI), Ultrasound, or other suitable imaging technique. The X-ray, CTA, MRA, phase-contrast MRI, or ultrasound images may be uploaded to or maintained on a storage platform, such as a cloud- or network-based storage platform, for further access and use.

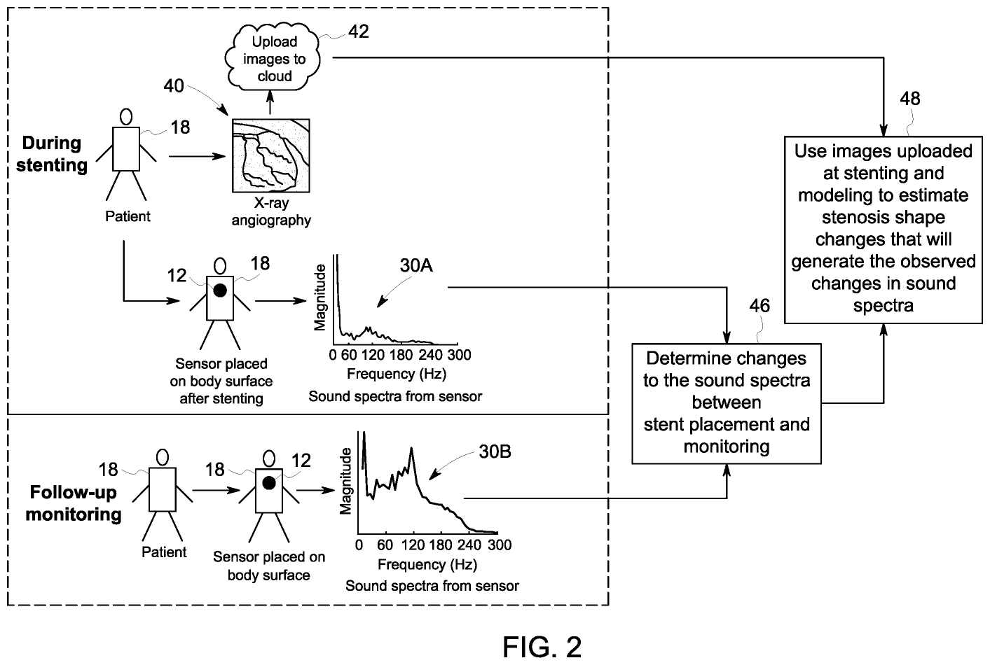

This process is shown diagrammatically in FIG. 2. As shown in the figure, in this example the patient 18 undergoes a stenting procedure (upper frame). As part of this process, X-ray image data (here shown as X-ray angiography image(s) 40) are acquired during deploying the stent. In the depicted example, the X-ray image(s) are stored in a suitable storage construct (such as being uploaded (step 42)) and stored in the cloud in the depicted example. X-ray angiography images 40 may augment pre-existing image data from the associated anatomy.

In addition, a sensor 12 of an acoustic sensing system 10 is used to collect acoustic data or signals 30, here depicted as a sound spectrum 30A contemporaneous or close to contemporaneously with the stenting procedure, such that the sound spectrum 30A corresponds to the structural and functional characteristics of the vasculature in question at the time of the procedure. By way of example, in the present stenting context, one or more sensors 12 are placed on the patient's chest wall after stenting and a baseline sound spectrum 30A is recorded. Signal processing techniques may be employed to isolate the sound generated from the coronary arteries from the other larger heart sounds. At a follow-up monitoring (bottom frame of FIG. 2), a sensor 12 may again be positioned on the patient, such as on the chest wall, and the sound spectrum (e.g., subsequent sound spectrum 30B) from the coronary arteries is again recorded. The subsequent sound spectrum 30B may be similarly processed to isolate the sounds of the vasculature of interest.

Using the uploaded or stored images 40, any other available imaging data of the anatomy, e.g. CTA and MRA images, a patient-specific computational model of sound propagation from the vasculature of interest at the time of stenting is generated and tuned to match the sound spectra at baseline (sound spectrum 30A). It is to be understood that in constructing this model not only are the stored images used to extract the vessel anatomy but also the blood flow in the vessels of interest. The flow in the vessel of interest could be obtained based on contrast dynamics information in stored X-ray/CTA images. It could also be obtained from stored phase-contrast MRI images, Doppler Ultrasound, or other suitable imaging techniques. The stored images could be two-, three-, or four-dimensional, the fourth dimension being time. Flow information extracted could be the volume flow rate or the velocity field in the vessel of interest as a function of time. In addition, the sensor measurements at baseline can be calibrated against the flow obtained from the baseline patient images and the calibrated sensor can be then used to predict the flow at subsequent time points in addition to the acoustic signature. To generate the computational model, the stored images are segmented to extract the vasculature of interest as well as the surrounding structures. Using the segmented vessel, transient hemodynamics calculations are conducted. Flow in the vessels of interest, obtained from the stored images, are used as boundary conditions. Pressure fluctuations, obtained on the vessel walls from the hemodynamics calculations, are then used as input for a linearized structural wave equation for the propagation of vibrations through the surrounding tissue. This describes a one-way coupled computational model. A two-way coupled approach can also be used, where the hemodynamics and the propagation of vibrations through the surrounding structures are solved at the same time. The generated computational model is then used to estimate the change in stenosis size needed to generate the observed differences in sound spectra between the baseline scan (sound spectrum 30A) and the one recorded at follow-up (sound spectrum 30B). These steps are illustrated in FIG. 2 as step 46, in which changes between the baseline sound spectrum 30A and subsequent sound spectrum 30B are determined and step 48, in which the images 40 are used to generate a model that can be used to estimate stenosis shape changes that will generate the observed sound spectra difference determined at step 46. In one embodiment, the change in stenosis size between baseline and follow-up is determined by solving an optimization problem. As mentioned before, a patient-specific computational model of sound propagation from the vasculature of interest is generated and this model is tuned to match the sound spectra at baseline. The vasculature of interest is then iteratively altered so that the error between the sound spectrum predicted by the computational model and the measured spectrum at the follow-up time is minimized. In another embodiment, the baseline computational model is used to generate a patient-specific library of acoustic signatures, with each acoustic signature corresponding to a unique stenosis shape and degree. The sound spectrum at follow-up is then compared against this library and the stenosis shape with the sound spectrum that is closest to the measured sound spectrum is selected. Though the present example relates to re-stenosis, as noted herein, the present approach may be more generally applied to monitoring a range of (if not all) vascular defects or irregularities based on acoustic signature data and prior patient images. The patient-specific computational model for sound propagation may be updated based on periodic, subsequently-acquired imaging data and corresponding acoustic propagation measurements acquired contemporaneously or close to contemporaneously with periodic acquisition of the imaging data. Updating the computational model for sound propagation improves its predictive capability.

In a further aspect, the present techniques may also be employed to provide a low-cost process for stratifying patients at risk of their first cardiac event (i.e., patients for whom prior screening data may not exist). By way of example, an implementation of this technique may be useful for detection (and subsequent therapy monitoring) of the presence of vulnerable plaques in high-risk individuals suspected of coronary artery disease. Approximately, 550,000 individuals in the United States have their first ischemic cardiac event each year without any a priori knowledge of heart disease. However, their risk factors (such as body habitus, sedentary lifestyle, smoking history, in vitro diagnostics, etc.) may indicate that they may be at risk for the disease. Of these, approximately 350,000 of the patients will not survive this first cardiac event. In practice, it may not be cost-effective to screen this patient cohort with medical imaging due to the time and expense involved. As such, a readily-available, low-cost, non-invasive method to first identify at-risk individuals, and then to monitor disease regression/progression in these individual longitudinally or during treatment would be of high value.

With this in mind, in one implementation, for a population where X-ray or CTA images may not be available (such as individuals previously untreated for heart disease), a population-derived model of a typical heart and coronary vasculature may instead be derived. One can then assume blockages in the population-derived coronary vessel models to characterize sound spectra signatures for classes of one or more stenosis (or other vascular structural features/anomalies), either in single-vessel or multi-vessel disease presentation. Population data can then be used to estimate prognostics or diagnostics (e.g., stenosis percentages) by correlating these sound spectra signatures to patient-specific measurements using sensors positioned on the patient's chest wall. In some embodiments, the population data could be augmented by low-cost, non-invasive ultrasound data acquired concurrently during the procedure to improve the relevance of the population data.

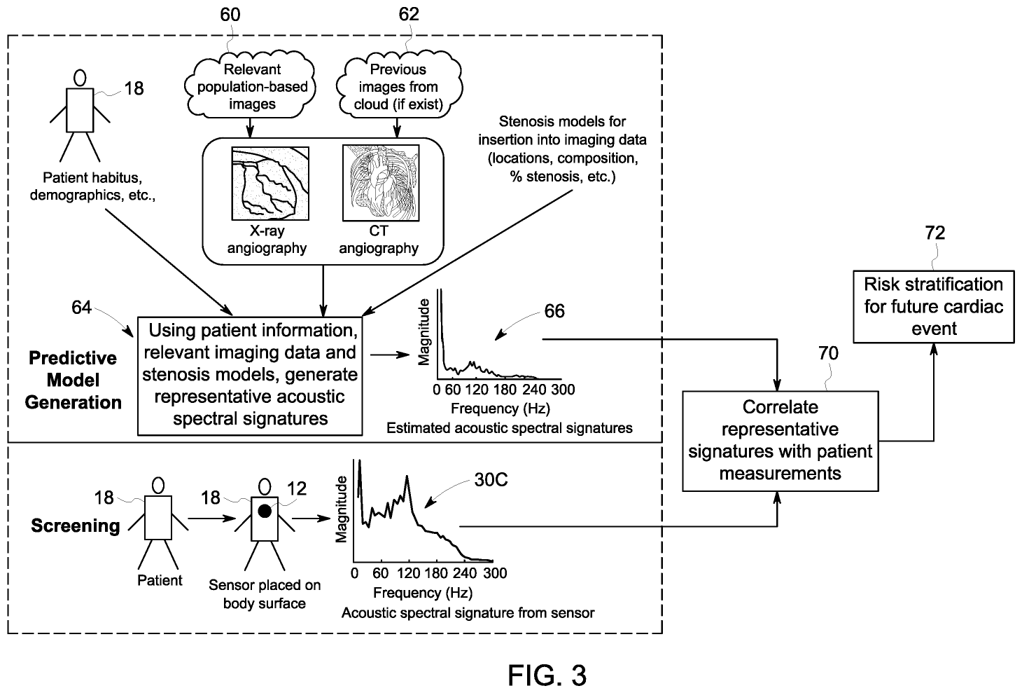

This process is shown diagrammatically in FIG. 3. As shown in the figure (top frame of FIG. 3) for predictive model generation, the patient 18 may be characterized by various factors (e.g., patient habitus, age, smoking history, in vitro diagnostics, demographics, and so forth). Some or all of these factors may be used to retrieve (step 60) relevant population-based vascular images (i.e., non-patient-specific images) from accessible data stores (X-ray, CT, MRI, Ultrasound, etc.). In addition to the population-based images, if patient-specific images are available, they may be retrieved (step 62) to augment the population-based images. To distinguish this implementation from the one illustrated in FIG. 1, the patient-specific images may not provide the high-fidelity vascular information available for the implementation illustrated in FIG. 1, since it is assumed that the patient has no known cardiovascular disease. For example, such scans may have been performed for other purposes, such for general thorax examination; although not providing high-fidelity vascular information, they may provide additional anatomical context relevant for developing the acoustic models. As stated above, concurrently acquired ultrasound data may be used to augment the population-based images. These images, along with patient factors and various stenosis models, are used to generate representative acoustic signatures corresponding to various vascular conditions 64, resulting in one or more acoustic spectral signatures 66. For example, acoustic spectral signatures 66 may be estimated by applying various solitary or multi-focal stenosis models to the retrieved image data based on patient information to generate signatures 66 for different stenosis locations, compositions, % stenosis, and so forth. The acoustic spectral signatures 66 may be generated a priori for various patient habitus and vascular conditions, and the patient information used to down-select relevant models. Alternatively, estimated acoustic signatures 66 may be discerned by acquiring longitudinal acoustic data from the general population, such as during normal yearly physical exams; stratifying the data based on one or more aforementioned features, e.g., patient habitus; tracking disease progression; and correlating acoustic signatures to disease progression, using one or more of standard regression, machine-learning, and/or deep-learning approaches.

At a screening examination (bottom frame of FIG. 3), a sensor 12 may be positioned on the patient 18, such as on the chest wall, and the sound spectrum (e.g., screening sound spectrum 30C) from the vasculature of interest (e.g., coronary arteries) is recorded. The screening sound spectrum 30C may be processed to isolate the sounds of the vasculature of interest.

At step 70, the measured screening sound spectrum 30C may be compared to or correlated with the representative acoustic spectral signatures 66 which correspond to or incorporate various structural irregularities or defects. Based on this comparison, the patient 18 may be risk stratified (step 72) (e.g., extremely high risk, high risk, moderate risk, low risk, etc.) for future cardiac events from cardiovascular anomalies, e.g., vascular defects in one or more coronary arteries or valvular defects. In this manner, a patient may be non-noninvasively characterized or categorized with respect to various vascular risks. Though the present example relates generally to cardiac events, the present approach may be more generally applied to detecting or predicting a range of (if not all) vascular defects or irregularities based on acoustic signature data.

Technical effects of the invention include, but are not limited to, the use of prior images acquired of the patient and stenosis acoustic signature to create a patient-specific model of sound propagation from the coronary arteries. This model is then used to detect the presence of disease and determine the degree of stenosis, using subsequently-acquired acoustic signals. In an alternate embodiment, population-based images are used to generate predictive data when a priori imaging information is not available and this data is used to characterize or categorize at-risk patients suspected of coronary artery disease, but without prior cardiac events.

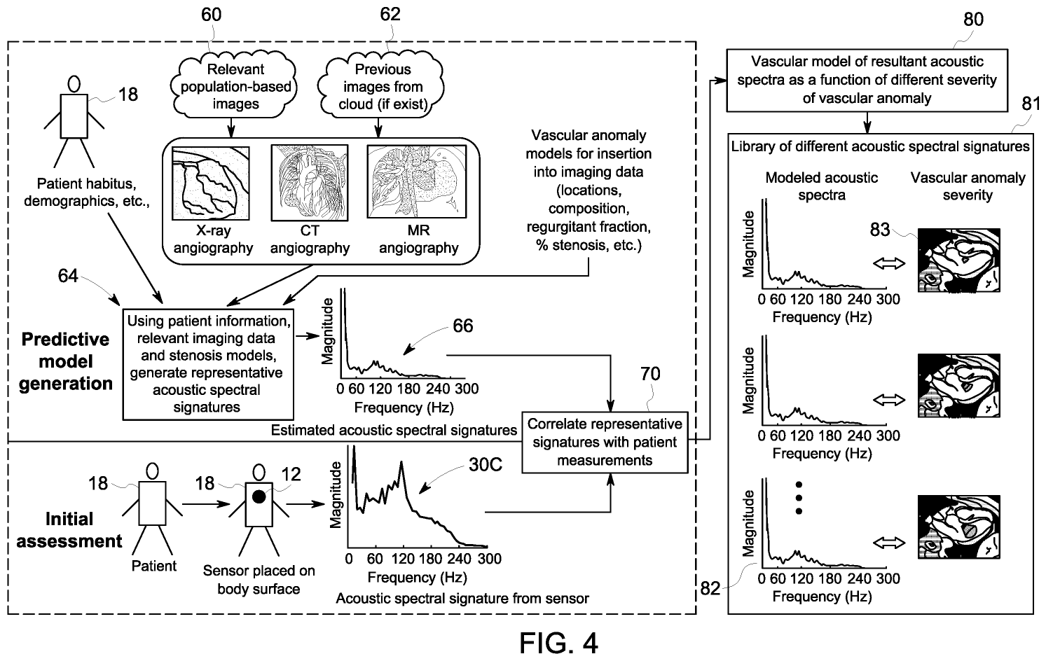

In another embodiment, illustrated in FIG. 4, the physician may choose to monitor the patient's progress and elect not to perform an interventional and therapeutic procedure to correct for the vascular anomaly if there is some risk to the therapeutic procedure. In such event, the patient 18 would have an initial workup that involves an imaging procedure (X-ray, CT, MR, or ultrasound) that maps the vascular structure and stores the information 62 that can be retrieved at a later time. With the sensor 12 placed on the patient 18, the acoustic spectral signal 30C is recorded. Specific modeling of the estimated acoustic spectral signal 66 is performed using the patient's data and also data from the population model 60, in a process similar to that discussed for the embodiment illustrated in FIG. 3 and described above. This approach allows the generating of a predictive model based on the patient-specific data over time. This predictive model allows the physician to predict the trajectory of change, based on the population data. In this manner, the physician will be able to determine the frequency of follow-up exams to better manage the patient progress. Parameters that are unique to the patient 18 are computed and stored such that the model-estimated acoustic spectral signal 66 matches with the actual recorded acoustic spectral signal 30C. These parameters are stored in the correlation model 70.

Subsequent to this, different estimated acoustic spectral signal signatures 82 can be generated by the vascular model 80 with different degrees of vascular changes or severity of the vascular anomaly 83, with each different vascular state having a unique acoustic spectral signature specific to this patient. These vascular signatures and the modeling of changes would have been previously validated using the population-based data 60. The patient can then have continuous or close-to-continuous monitoring with the measured acoustic spectral signal matched against the library of acoustic spectral signatures 81 specific to that patient. In this manner, the physician can be alerted as to any significant change in the hemodynamic significance of the vascular anomaly of the patient and intervene accordingly.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. For example, in the descriptions above, the acoustic signals have been characterized by spectral content. The acoustic signals also can be characterized by their time-domain representations or signatures. The relationship between time-domain and spectral-domain representations is known in the art, and the methods described herein equally apply to time-domain representations or signatures. Furthermore, in the description above, acoustic signals are used to explain the multiple embodiments. However, the use of the term “acoustic signal” is not meant to be limiting, and signals with spectral or temporal components outside of the audible range are also contemplated.

1. A method for monitoring vascular health of a patient, comprising:
acquiring patient-specific imaging data of a vascular region of interest;
acquiring acoustic data of the vascular region of interest contemporaneous or close to contemporaneous with acquisition of the patient-specific imaging data;
creating a patient-specific model of sound propagation for the vascular region of interest using the patient-specific imaging data and the acoustic data;
identifying differences between the acoustic data acquired contemporaneous or close to contemporaneous with the patient-specific imaging data and a subsequent acoustic data set; and
processing the differences using the patient-specific model to estimate a change in vascular structure or function of the patient.

2. The method of claim 1, wherein the patient-specific imaging data used to generate the patient-specific model includes quantitative vascular flow information to supplement the patient-specific imaging data.

3. The method of claim 2, wherein the quantitative vascular flow information includes velocity flow data or volume flow rate data as a function of time.

4. The method of claim 2, wherein the quantitative flow information and the patient-specific imaging data is two-dimensional or three-dimensional spatially.

5. The method of claim 4, wherein the two-dimensional or three-dimensional spatial data further comprises a temporal dimension.

6. The method of claim 1, where estimating a change in vascular structure or function of the patient involves:
generating a patient-specific library of acoustic signatures using the patient-specific model of sound propagation and different states of vascular anomaly; and
comparing the subsequent acoustic data set against estimates associated with the patient-specific library of acoustic signatures to find a vascular anomaly state that best matches the subsequent acoustic data.

7. The method of claim 1 where an indication or alert is generated if a change in hemodynamic significance due to the change in vascular structure is sufficient for a medical action to be taken.

8. The method of claim 6, further comprising:
performing periodic imaging to acquire data of underlying vascular architecture of the patient;
generating or measuring a corresponding acoustic signature contemporaneously or close to contemporaneously with the acquisition of the patient-specific imaging data;
updating the patient-specific model of sound propagation with new data associated with the acoustic signature and the patient-specific imaging data to improve accuracy of the estimates of the patient-specific library of acoustic signatures.

9. The method of claim 6, further comprising:
acquiring on a continuous basis the acoustic data and communicating the acoustic data to a central station;
comparing the acoustic data against the estimates of the patient-specific library of acoustic signatures to determine if the change in the hemodynamic significance of the vascular anomaly exceeds a threshold for hemodynamic significance that prompts medical attention.

10. A method for characterizing vascular risk of a patient, comprising:
acquiring population-based data of a vascular region of interest;
generating one or more estimated acoustic spectral signatures based on the population-based data;
comparing or correlating the estimated acoustic spectral signatures with screening acoustic data; and
based upon the comparison or correlation, characterizing or categorizing a risk state of the patient.

11. The method of claim 10, wherein the population-based data used to generate the one or more estimated spectral signatures comprises at least one of population-based images used to derive an acoustic model and longitudinal acoustic data acquired from the general population.

12. The method of claim 10, comprising data associated with population-based images augmented by ultrasound data acquired concurrently during a procedure to improve relevance of the data.

13. An analysis system for monitoring vascular health of a patient, comprising:
a memory encoding processor-executable routines; and
a processing component configured to access the memory and execute the processor-executable routines, wherein the routines, when executed by the processing component, cause the processing component to perform actions comprising:
acquiring patient-specific imaging data of a vascular region of interest;
acquiring acoustic data of the vascular region of interest contemporaneous or close to contemporaneous with acquisition of the patient-specific imaging data;
creating a patient-specific model of sound propagation for the vascular region of interest using the patient-specific imaging data and the acoustic data;
identifying differences between the acoustic data acquired contemporaneous or close to contemporaneous with the patient-specific imaging data and a subsequent acoustic data set; and
processing the differences using the patient-specific model to estimate a change in vascular structure or function of the patient.

14. The analysis system of claim 13, wherein the patient-specific imaging data used to generate the patient-specific model includes quantitative vascular flow information to supplement the patient-specific imaging data.

15. The analysis system of claim 14, wherein the quantitative vascular flow information includes velocity flow data or volume flow rate data as a function of time.

16. The analysis system of claim 14, wherein the quantitative flow information and the patient-specific imaging data is two-dimensional or three-dimensional spatially.

17. The analysis system of claim 16, wherein the two-dimensional or three-dimensional spatial data further comprises a temporal dimension.

18. The analysis system of claim 13, where estimating a change in vascular structure or function of the patient involves:
generating a patient-specific library of acoustic signatures using the patient-specific model of sound propagation and different states of vascular anomaly;
and comparing the subsequent acoustic data set against estimates associated with the patient-specific library of acoustic signatures to find a vascular anomaly state that best matches the subsequent acoustic data.

19. The analysis system of claim 13 where an indication or alert is generated if a change in hemodynamic significance due to the change in vascular structure is sufficient for a medical action to be taken.

20. The analysis system of claim 18, further comprising:
performing periodic imaging to acquire data of underlying vascular architecture of the patient;
generating or measuring a corresponding acoustic signature contemporaneously or close to contemporaneously with the acquisition of the patient-specific imaging data;
updating the patient-specific model of sound propagation with new data associated with the acoustic signature measurement and the patient-specific imaging data to improve the accuracy of the estimates of the patient-specific library of acoustic signatures.

21. The analysis system of claim 18, further comprising:
acquiring on a continuous basis the acoustic data and communicating the acoustic data to a central station;
comparing the acoustic data against the estimates of the patient-specific library of acoustic signatures to determine if the change in the hemodynamic significance of the vascular anomaly exceeds a threshold for hemodynamic significance that prompts medical attention.
